(19) United States
(12) Patent Application Publication
(10) Pub. No.: US 2015/0306349 A1
(43) Pub. Date: Oct. 29, 2015

(54) VALVED CATHETER ASSEMBLIES AND RELATED METHODS

(71) Applicant: B. BRAUN MELSUNGEN AG, Melsungen (DE)

(72) Inventor: Olivier Bonnal, Melsungen (DE)

(21) Appl. No.: 14/698,529

(22) Filed: Apr. 28, 2015

Related U.S. Application Data
(60) Provisional application No. 61/985,861, filed on Apr. 29, 2014.

(51) Int. Cl. A61M 25/00

(52) U.S. Cl. CPC A61M 25/0097

(57) ABSTRACT

The valved catheter assemblies described herein provide several advantages, including low cracking pressure for fluid infusion, greater cracking pressure for fluid aspiration, and reduction or elimination of dead space. In some embodiments, a valve of the catheter assembly has a conical shape, which provides different cracking pressures for infusion and aspiration. Also in some embodiments, a valve of the catheter assembly may include peripheral slits that enable fluid to flow around the perimeter of the valve during infusion, which helps to flush out fluid from around the valve.

### TECHNICAL FIELD

The present embodiments relate to aspiration/infusion devices, such as intravenous (“IV”) catheters. In particular, the present embodiments relate to flushable peripheral intravenous catheter assemblies having features to enable selective activation of fluid flow through the catheter assemblies.

### BACKGROUND

Catheters are commonly used for infusing fluids, such as saline, various medicaments, and total parenteral nutrition, into a patient's vasculature. Catheters are also commonly used for withdrawing blood from a patient, or monitoring various parameters of the patient's vascular system.

Catheters and/or needles are typically coupled to a catheter adapter, also referred to as a catheter hub, to enable attachment of IV tubing to the catheter. Thus, following placement of the catheter or needle into the patient's vasculature, the catheter adapter is coupled to a fluid source via a section of IV tubing. In order to verify proper placement of the needle and/or catheter in the blood vessel, the clinician generally confirms that there is “flashback” of blood in a flashback chamber of the catheter assembly.

Once proper placement of the catheter is confirmed, the clinician then attaches the catheter adapter to IV tubing. To prevent undesirable exposure to blood, the clinician typically must maintain pressure on the patient's vein while simultaneously coupling the catheter adapter to the IV tubing. This procedure can be awkward. A common, but undesirable, practice is to permit blood to temporarily and freely flow from the catheter adapter while the clinician locates and couples the IV tubing to the catheter adapter. Another common practice is to attach the catheter adapter to the IV tubing prior to placing the needle or catheter into the vein of the patient. While this method may prevent undesirable exposure to blood, positive pressure within the W line may also prevent desirable flashback.

SUMMARY

The various embodiments of the present valved catheter assemblies and related methods have several features, no single one of which is solely responsible for their desirable attributes. Without limiting the scope of the present embodiments as expressed by the claims that follow, their more prominent features now will be discussed briefly. After considering this discussion, and particularly after reading the section entitled “Detailed Description,” one will understand how the features of the present embodiments provide the advantages described herein.

One embodiment of the present catheter assemblies comprises a catheter hub defining an interior cavity. The catheter assembly further comprises a catheter extending distally from the catheter hub, the catheter including a catheter lumen. The catheter assembly further comprises a valve disposed within the interior cavity of the catheter hub. The valve includes a conically shaped diaphragm with at least one slit defining at least two flaps. The catheter assembly further comprises a needle hub engaging a proximal end of the catheter hub. The catheter assembly further comprises a needle extending distally from the needle hub, through the catheter hub, through the valve, and through the catheter lumen, the needle having a sharp distal tip.

An apex of the diaphragm may lie in a plane distal of a plane defined by a base of the diaphragm. When the needle extends through the valve, a gap between the valve flaps may have sufficient width to allow passage of gaseous particles but insufficient width to allow passage of liquid particles. After the needle is withdrawn from the valve, the valve may have a first cracking pressure in the proximal-to-distal flow direction and a second cracking pressure in the distal-to-proximal flow direction, and the first and second cracking pressures may be different. The second cracking pressure may be greater than the first cracking pressure. The second cracking pressure may be greater than a maximum blood pressure of a patient in which the catheter assembly is placed. The valve may include an annular flange at a periphery of the valve. The flange may extend proximally of the diaphragm. A proximal face of the valve may include a circular lip positioned radially inward of the flange. The catheter hub may include a proximal hub element and a distal hub element formed as discrete components and fitted together. A periphery of the valve may be interposed between the proximal hub element and the distal hub element of the catheter hub. A proximal face of the valve may include a circular lip that bears against an annular distal face of the proximal hub element of the catheter hub. The valve may include an annular flange at a periphery of the valve, and the annular flange may overlap and surround a distal portion of the proximal hub element of the catheter hub. The annular flange may be interposed between the proximal hub element and the distal hub element of the catheter hub as measured along the direction perpendicular to a longitudinal axis of the catheter. The diaphragm may include three slits extending radially outward from a center of the diaphragm and defining three flaps. The diaphragm may further comprise a plurality of reinforcing ribs located, at least in part, on the flaps. The catheter assembly may further comprise a needle tip protector disposed within the interior cavity of the catheter hub.

Another embodiment of the present catheter assemblies comprises a catheter hub defining an interior cavity. The catheter assembly further comprises a catheter extending distally from the catheter hub. The catheter includes a catheter lumen. The catheter assembly further comprises a valve disposed within the interior cavity of the catheter hub. The valve includes a diaphragm with at least one slit defining at least two flaps. The valve has a first cracking pressure in the proximal-to-distal flow direction and a second cracking pressure in the distal-to-proximal flow direction, and the first and second cracking pressures are different.

The second cracking pressure may be greater than the first cracking pressure. The second cracking pressure may be greater than a maximum blood pressure of a patient in which the catheter assembly is placed. A central portion of the diaphragm may extend distally with respect to a periphery of the diaphragm. The valve may include an annular flange at a periphery of the valve. The flange may extend proximally of the diaphragm. A proximal face of the valve may include a circular lip positioned radially inward of the flange. The catheter hub may include a proximal hub element and a distal hub element formed as discrete components and fitted together. A periphery of the valve may be interposed between the proximal hub element and the distal hub element of the catheter hub. A proximal face of the valve may include a circular lip that bears against an annular distal face of the proximal hub element of the catheter hub. The valve may include an annular flange at a periphery of the valve, and the annular flange may overlap and surround a distal portion of the proximal hub element of the catheter hub. The annular flange may be interposed between the proximal hub element and the distal hub element of the catheter hub as measured along the direction perpendicular to a longitudinal axis of the catheter. The diaphragm may include three slits extending radially outward from a center of the diaphragm and defining three flaps. The diaphragm may further comprise a plurality of reinforcing ribs located, at least in part, on the flaps.

Aspects of the present disclosure include a catheter assembly which can comprise a catheter hub defining an interior cavity, a catheter extending distally from the catheter hub, the catheter including a catheter lumen, a valve disposed within the interior cavity of the catheter hub, the valve including a conically shaped diaphragm with at least one slit defining at least two flaps, a needle hub engaging a proximal end of the catheter hub, and a needle extending distally from the needle hub, through the catheter hub, through the valve, and through the catheter lumen, the needle having a sharp distal tip.

An apex of the diaphragm can lie in a plane distal of a plane defined by a base of the diaphragm.

When the needle extends through the valve, a gap between the valve flaps can have sufficient width to allow passage of gaseous particles but can have insufficient width to allow passage of liquid particles.

After the needle is withdrawn from the valve, the valve can have a first cracking pressure in the proximal-to-distal flow direction and a second cracking pressure in the distal-to-proximal flow direction, and the first and second cracking pressures can be different.

The second cracking pressure can be greater than the first cracking pressure.

The second cracking pressure can be greater than a maximum blood pressure of a patient in which the catheter assembly is placed.

The valve can include an annular flange at a periphery of the valve.

The flange can extend proximally of the diaphragm.

A proximal face of the valve can include a circular lip positioned radially inward of the flange.

The catheter hub can include a proximal hub element and a distal hub element formed as discrete components and fitted together.

A periphery of the valve can be interposed between the proximal hub element and the distal hub element of the catheter hub.

A proximal face of the valve can include a circular lip that bears against an annular distal face of the proximal hub element of the catheter hub.

The valve can include an annular flange at a periphery of the valve, and the annular flange can overlap and surround a distal portion of the proximal hub element of the catheter hub.

The annular flange can be interposed between the proximal hub element and the distal hub element of the catheter hub as measured along the direction perpendicular to a longitudinal axis of the catheter.

The diaphragm can include three slits extending radially outward from a center of the diaphragm and define three flaps.

The diaphragm further can comprise a plurality of reinforcing ribs located, at least in part, on the flaps.

The catheter assembly can further comprise a needle tip protector disposed within the interior cavity of the catheter hub.

Another aspect of the present disclosure includes a catheter assembly which can comprise a catheter hub defining an interior cavity, a catheter extending distally from the catheter hub, the catheter including a catheter lumen, and a valve disposed within the interior cavity of the catheter hub, the valve including a diaphragm with at least one slit defining at least two flaps, wherein the valve has a first cracking pressure in the proximal-to-distal flow direction and a second cracking pressure in the distal-to-proximal flow direction, and the first and second cracking pressures are different.

The second cracking pressure can be greater than the first cracking pressure.

The second cracking pressure can be greater than a maximum blood pressure of a patient in which the catheter assembly is placed.

A central portion of the diaphragm can extend distally with respect to a periphery of the diaphragm.

The valve can include an annular flange at a periphery of the valve.

The flange can extend proximally of the diaphragm.

A proximal face of the valve can include a circular lip positioned radially inward of the flange.

The catheter hub can include a proximal hub element and a distal hub element formed as discrete components and fitted together.

A periphery of the valve can be interposed between the proximal hub element and the distal hub element of the catheter hub.

A proximal face of the valve can include a circular lip that bears against an annular distal face of the proximal hub element of the catheter hub.

The valve can include an annular flange at a periphery of the valve, and the annular flange can overlap and surround a distal portion of the proximal hub element of the catheter hub.

The annular flange can be interposed between the proximal hub element and the distal hub element of the catheter hub as measured along the direction perpendicular to a longitudinal axis of the catheter.

The diaphragm can include three slits extending radially outward from a center of the diaphragm and defining three flaps.

The diaphragm can further comprise a plurality of reinforcing ribs located, at least in part, on the flaps.

### BRIEF DESCRIPTION OF THE DRAWINGS

The various embodiments of the present valved catheter assemblies and related methods now will be discussed in detail with an emphasis on highlighting the advantageous features. These embodiments depict the novel and non-obvious valved catheter assemblies and related methods shown in the accompanying drawings, which are for illustrative purposes only. These drawings include the following figures, in which like numerals indicate like parts:

FIG. 1 is a side cross-sectional view of one embodiment of the present valved catheter assemblies;

FIG. 2 is a partial cross-sectional front perspective view of the valve of the valved catheter assembly of FIG. 1;

FIG. 3 is a detail view of a portion of the valved catheter assembly of FIG. 1, as indicated by the box 3-3 in FIG. 1;

FIG. 4 is a side cross-sectional view of the valved catheter assembly of FIG. 1 with some components removed;

FIG. 5 is a detail view of a portion of the valved catheter assembly of FIG. 4, as indicated by the box 5-5 in FIG. 4;

FIG. 5A is a schematic side view of the valve of the valved catheter assembly of FIG. 1;

FIG. 6 is a side cross-sectional view of another embodiment of the present valved catheter assemblies;

FIG. 7 is a front view of another embodiment of a valve configured for use with the present valved catheter assemblies; and

FIG. 8 is a side cross-sectional view of another embodiment of the present valved catheter assemblies, including the valve of FIG. 7 and illustrating the configuration of the assembly during an infusion procedure.

### DETAILED DESCRIPTION

The following detailed description describes the present embodiments with reference to the drawings. In the drawings, reference numbers label elements of the present embodiments. These reference numbers are reproduced below in connection with the discussion of the corresponding drawing features.

The embodiments of the present valved catheter assemblies and related methods are described below with reference to the figures. These figures, and their written descriptions, indicate that certain components of the apparatus are formed integrally, and certain other components are formed as separate pieces. Those of ordinary skill in the art will appreciate that components shown and described herein as being formed integrally may in alternative embodiments be formed as separate pieces. Those of ordinary skill in the art will further appreciate that components shown and described herein as being formed as separate pieces may in alternative embodiments be formed integrally. Further, as used herein the term integral describes a single unitary piece.

The present embodiments include methods of using a valved catheter assembly. Some of these embodiments may be performed in connection with treating a human and/or animal body. Others of these embodiments may be performed independently of a human and/or animal body, such as for purposes of testing or demonstration of the valved catheter assembly. Accordingly, the present embodiments pertaining to methods of using a valved catheter assembly should not be construed as limited to methods of treating a human and/or animal body.

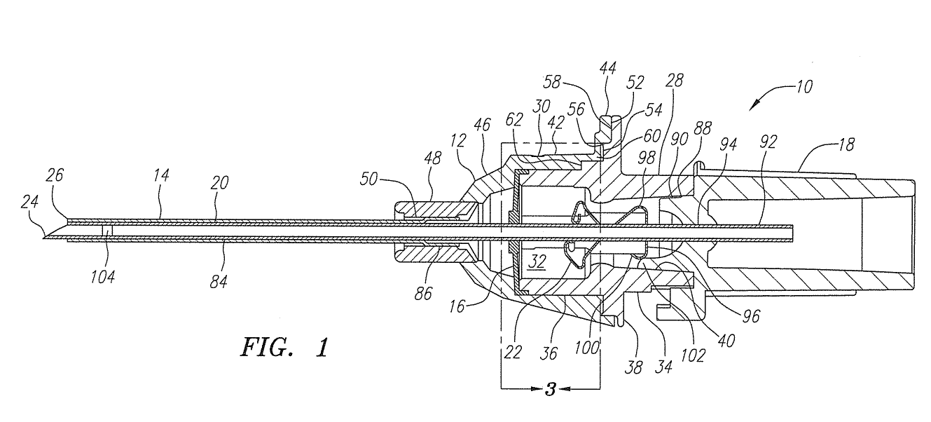

FIGS. 1-5 illustrate one embodiment of the present valved catheter assemblies 10. The assembly 10 is configured for infusing fluids, such as saline, various medicaments, total parenteral nutrition, etc., into a patient's vasculature, withdrawing blood from a patient, and monitoring various parameters of the patient's vascular system. With reference to FIG. 1, in some embodiments the assembly 10 includes a catheter hub 12, a catheter 14, a valve 16, a needle hub 18, a needle 20, and a needle tip protector 22. The needle 20 includes a sharp distal tip 24. In the pre-placement or ready for use configuration of FIG. 1, the sharp distal tip 24 is exposed from a distal end 26 of the catheter 14. The assembly 10 may include a removable cap (not shown) that extends over the needle 20 and at least partially over the catheter hub 12 in the pre-placement configuration to cover the sharp distal tip 24 and thereby reduce the likelihood of needlestick.

With continued reference to FIG. 1, the catheter hub 12 comprises a two-part hub body including a proximal hub element 28 and a distal hub element 30 that together define an interior cavity 32. In the illustrated embodiment, the proximal and distal hub elements 28, 30 are formed as discrete components and fitted together. However, the illustrated embodiment is just one example and is not limiting. The proximal and distal hub elements 28, 30 may be secured to one another by any appropriate means, such as a friction fit, adhesive, welding, etc.

The proximal hub element 28 includes a proximal annular portion 34, a distal annular portion 36, and an outwardly extending radial flange 38 adjacent the junction of the proximal and distal annular portions 34, 36. The proximal annular portion 34 forms a connecting section for receiving the needle hub 18, and for receiving another component, such as a syringe tip or an IV tubing adapter (not shown), after the needle hub 18 is removed from the catheter hub 12, as described below. Thus, in the illustrated embodiment, the inner surface 40 of the proximal annular portion 34 includes a conical female Luer taper.

The distal hub element 30 includes a proximal annular portion 42, an outwardly extending radial flange 44 at the proximal end of the proximal annular portion 42, a tapered portion 46 extending distally from the distal end of the proximal annular portion 42, and a distal annular portion 48 extending distally from the distal end of the tapered portion 46. The proximal annular portion 42 forms a receiving section for receiving the distal annular portion 36 of the proximal hub element 28. The distal annular portion 48 includes a passage 50 for holding the catheter 14.

With continued reference to FIG. 1, the distal annular portion 36 of the proximal hub element 28 is received within the proximal annular portion 42 of the distal hub element 30 with the flanges 38, 44 of the two hub elements 28, 30 abutting one another. A proximal face 52 of the flange 44 on the distal hub element 30 includes an annular recess 54 that matingly receives an annular raised portion 56 on the distal face 58 of the flange 38 on the proximal hub element 28. The proximal hub element 28 further includes a key 60 that is received within a keyway 62 in the distal hub element 30. The mating key-in-keyway engagement ensures that the proximal and distal hub elements 28, 30 are properly rotationally aligned with one another. However, the illustrated key-in-keyway engagement is just one example, and is not limiting. In an example, the depth of the distal annular portion 36 into the distal hub element may be adjusted or modified to control the placement location of the valve 16 within the catheter hub, as further discussed below. In other examples, the valve 16 is placed into a singularly formed catheter hub.

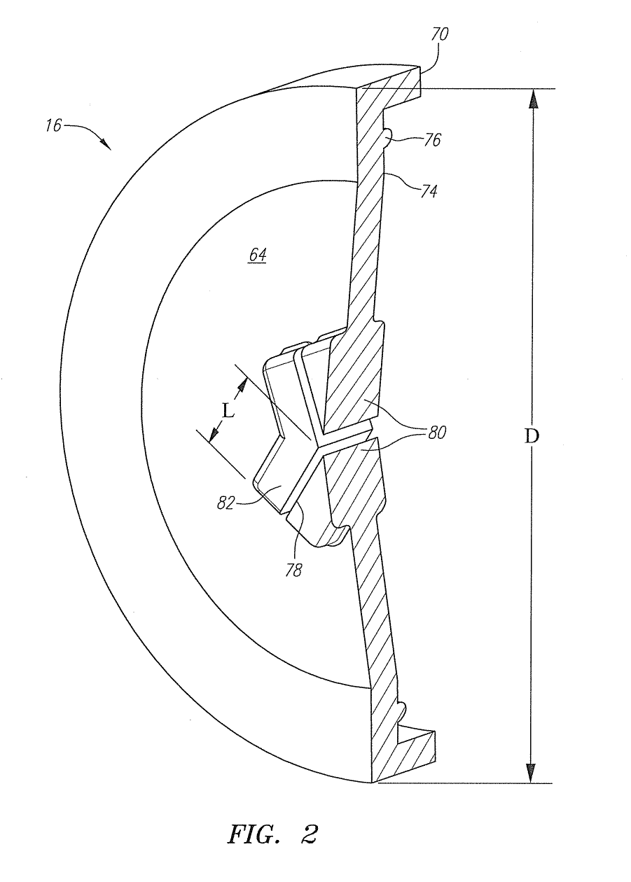

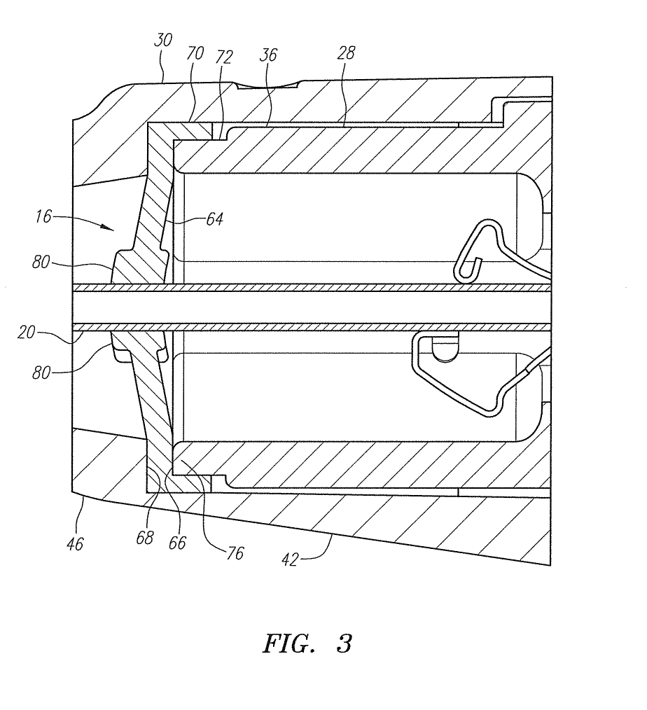

With continued reference to FIG. 1, the valve 16 is disposed within the interior cavity 32 of the catheter hub 12. FIG. 2 is a partial cross-sectional front perspective view of the valve 16, and FIG. 3 is a detail view of the portion of the catheter assembly 10 of FIG. 1 indicated by the box 3-3 in FIG. 1, which includes the valve 16. With reference to FIGS. 2 and 3, the valve 16 includes a diaphragm or valve body 64, a periphery of which is interposed between the proximal and distal hub elements 28, 30 (FIG. 3), more specifically between a distal face 66 of the proximal hub element 28 and a proximal facing interior annular shoulder 68 of the distal hub element 30 at the junction of the proximal annular portion 42 and the tapered portion 46. With reference to FIG. 2, the valve 16 includes an annular flange 70 at a periphery of the diaphragm 64. With reference to FIG. 3, the flange 70 extends proximally from the diaphragm 64, and overlaps and surrounds a short length of the distal annular portion 36 of the proximal hub element 28 of the catheter hub 12 such that the annular flange 70 is interposed along the radial direction between the proximal and distal hub elements 28, 30 of the catheter hub 12. An outer surface of the distal annular portion 36 includes an annular recess 72 that receives the overlapping valve flange 70. The valve 16 may be held in place by compression between the mating hub elements 28, 30. Alternatively, or in addition, the valve 16 may be held in place by securing the valve 16 to either or both of the proximal and distal hub elements 28, 30 using adhesives, welding, etc.

With reference to FIG. 2, a proximal face 74 of the valve 16 includes a circular lip 76 positioned radially inward of the flange 70. With reference to FIG. 3, the lip 76 bears against the distal face 66 of the proximal hub element 28. The compressive force on the lip 76 deforms the lip 76, thereby increasing the efficacy of the liquid tight seal at the interface between the valve 16 and the proximal hub element 28.

The valve 16 preferably comprises a flexible and resilient medical grade material that is capable of forming a fluid tight seal at an interface between two components (where the valve 16 may be one of the components). For example, and without limitation, the valve 16 may comprise silicone or other medical grade polymer material.

With reference to FIG. 2, the diaphragm 64 includes slits 78 that define edges of flaps 80. In the illustrated embodiment, the diaphragm 64 includes three slits 78 and three flaps 80. However, the illustrated embodiment is just one example and is not limiting. The slits 78 converge at the center of the diaphragm 64 and are all equally spaced by about 120°. On either side of each slit 78, the edges of each flap 80 include reinforcing ribs 82 comprising areas of increased thickness of the diaphragm 64. The ribs 82 increase the flaps' 80 rigidity. In some embodiments, the ribs 82 are the same material as the diaphragm 64. For example, the ribs 82 may be formed as a unitary component of the diaphragm 64. In other embodiments, the ribs 82 may be made of the same material or a different material as the diaphragm 64 and joined to the diaphragm 64, such as with adhesive, welding, or in any other manner.

With reference to FIG. 1, the catheter 14 extends distally from the catheter hub 12 and includes a catheter lumen 84. A proximal end 86 of the catheter 14 is disposed within the passage 50 of the distal catheter hub element 30. The catheter 14 and the catheter hub 12 may be secured to one another by any appropriate means, such as a friction fit, adhesive, insert molding, etc.

The needle hub 18 engages a proximal end of the catheter hub 12. A connection between the needle hub 18 and the catheter hub 12 is such that the needle hub 18 may be deliberately disengaged from the catheter hub 12 and may include a friction fit. In the illustrated embodiment, a distal end of the needle hub 18 comprises a nose section 88 that is received within the proximal end of the proximal catheter hub element 28. The nose section 88 includes a conical Luer taper on its outer surface 90 that mates with the conical Luer taper on the inner surface 40 of the proximal catheter hub element 28. The needle hub 18 and/or the catheter hub 12 may include one or more mating securing elements, such as detents, latches, clasps, etc. (not shown) to reinforce the connection between the needle hub 18 and the catheter hub 12.

With further reference to FIG. 1, the needle 20 extends distally from the needle hub 18, through the catheter hub 12, through the valve 16, and through the catheter lumen 84. The proximal end 92 of the needle 20 is held within an opening 94 in the distal portion of the needle hub 18 by any appropriate means, such as a friction fit, adhesive, insert molding, etc. With reference to FIG. 3, the needle 20 extending through the valve 16 separates the valve flaps 80 from one another. In this configuration, narrow gaps between adjacent valve flaps 80 enable air to pass through the valve 16, but not liquid. Stated another way, when the needle 20 extends through the valve 16, a gap between adjacent valve flaps 80 has sufficient width to allow passage of gaseous particles but has insufficient width to allow passage of liquid particles. The valve 16 is thus advantageously configured to vent air during a venipuncture procedure, thereby enabling blood flashback, without allowing blood to leak through the valve 16. Blood thus does not contaminate any of the components located proximal of the valve 16, including the catheter hub 12, the needle hub 18, and the needle tip protector 22. Preventing blood contamination of these components reduces the risk of transmission of bloodborne pathogens.

With reference to FIG. 1, the interior cavity 32 of the catheter hub 12 receives the needle tip protector 22. The needle tip protector 22 covers the sharp distal tip 24 of the needle 20 to prevent needlesticks after the needle hub 18 is withdrawn from the catheter hub 12, as described below. The needle tip protector 22 includes a proximal wall 96 defining an opening through which the needle 20 passes, and a pair of legs 98, 100 extending distally from opposite ends of the proximal wall 96. The interior cavity 32 of the catheter hub 12 and/or the distal end of the needle hub 18 may include features that engage and support the needle tip protector 22. For example, the distal end of the needle hub 18 includes a recess 102 that receives one end of the proximal wall 96 of the needle tip protector 22. In alternative embodiments, the needle tip protector 22 may be omitted.

Following successful venipuncture, the needle 20 is withdrawn from the catheter assembly 10, leaving the catheter 14 disposed in the vein. To disengage the needle hub 18 from the catheter hub 12, the clinician holds the catheter hub 12 with one hand while pulling back on the needle hub 18 with the opposite hand. As the needle 20 withdraws from the catheter hub 12, the recess 102 at the distal end of the needle hub 18 engages the tip protector 22 to prevent proximal movement of the tip protector 22 with respect to the catheter hub 12. Eventually, a change in profile 104 near the distal tip 24 of the needle 20 engages the outer circumference of the opening in the proximal wall 96 of the tip protector 22 so that the needle 20 pulls the tip protector 22 proximally as the needle 20 withdraws from the catheter hub 12. At approximately the same time as the change in profile 104 of the needle 20 engages the proximal wall 96 of the tip protector 22, the distal tip 24 of the needle 20 passes between the legs 98, 100 of the tip protector 22. The legs 98, 100, which are biased toward one another, pivot radially inward so that the tip protector 22 disengages the catheter hub 12. The tip protector 22 then withdraws from the catheter hub 12 along with the needle 20 while the legs 98, 100 of the tip protector 22, and more specifically the distal end walls on the legs, block the return path of the needle tip 24. The sharp tip 24 is thus enclosed within the tip protector 22 to prevent needlesticks.

The change in profile 104 of the needle 20 may comprise, for example, a radial projection, which may be formed by lightly crimping the needle 20. In other embodiments, the change in profile 104 may comprise a sleeve, a notch, a material buildup on the shaft of the needle 20, etc.

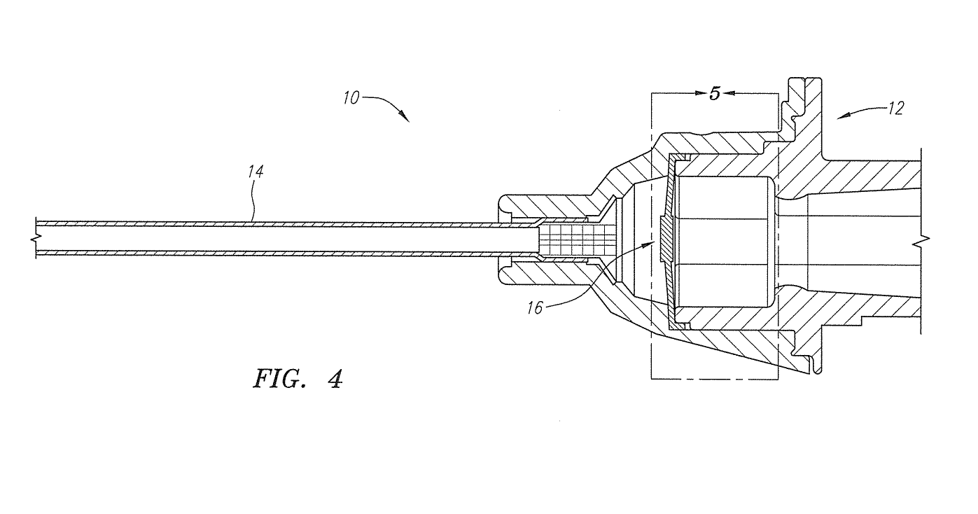

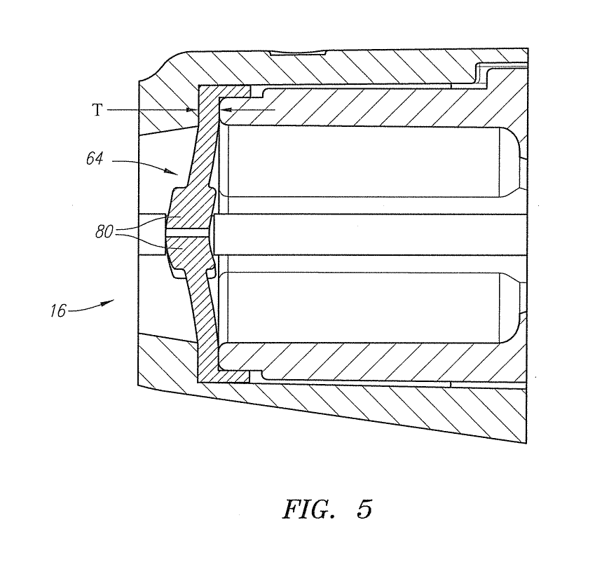

FIG. 4 illustrates the present catheter assembly 10 after the needle 20 is withdrawn from the catheter 14, and after the needle hub 18 is withdrawn from the catheter hub 12. FIG. 5 is a detail view of the portion of FIG. 4 indicated by the box 5-5 in FIG. 4. With reference to FIG. 5, after the needle 20 is withdrawn from the valve 16, the valve flaps 80 move toward one another due to the elasticity of the valve material, thereby closing the gaps between adjacent valve flaps 80. The configuration of the valve 16 in FIG. 5 thus illustrates the nondeformed or unstressed condition of the valve 16. In this configuration, the valve 16 forms an airtight seal that prevents movement of air and liquid through the catheter assembly 10 in either direction until the cracking pressure of the valve 16 is reached, as described below.

With continued reference to FIG. 5, in its nondeformed or unstressed condition the diaphragm 64 of the valve 16 is conically shaped. With reference to FIG. 5A, which is a schematic side view of the valve 16, an apex 106 of the diaphragm 64 lies in a plane 108 distal of a plane 110 defined by a base 112 of the diaphragm 64. This geometry creates differential cracking pressures for the valve 16. More specifically, the nondeformed valve 16 has a first cracking pressure in the proximal-to-distal flow direction FP→D (FIG. 5A) and a second cracking pressure in the distal-to-proximal flow direction FD→P. The first cracking pressure is lower than the second cracking pressure, because pressure applied to the distal face 114 of the diaphragm 64 tends to force the flaps 80 closer to one another, which only increases the strength of the seal. By contrast, pressure applied to the proximal face 116 of the diaphragm 64 tends to force the flaps 80 apart, and the conical shape of the diaphragm 64 provides greater surface area on the proximal face 116 as compared to a diaphragm that is shaped like a flat disk, thereby increasing the magnitude of the valve opening force for a given fluid pressure. And, the surface area of the proximal face 116 of the diaphragm 64 increases as the opening angle Θ of the cone decreases. Thus, the magnitude of the cracking pressure(s) of the diaphragm 64 can be tailored by varying the opening angle Θ of the cone. In certain embodiments, the opening angle Θ may be in the range from about 120° to about 175° with 180° being flush or flat. In some embodiments, the opening angle Θ is from about 150° to about 170°.

Preferably, the first cracking pressure (in the proximal-to-distal flow direction F→D) is close to zero. The valve 16 thus provides very low resistance to infusion of fluids through the catheter assembly 10. In certain embodiments, the first cracking pressure may be in the range from about 0 mmH2O to about 10 mmH2O, for example, from about 1 mmH2O to about 8 mmH2O.

Preferably, the second cracking pressure (in the distal-to-proximal flow direction FD→P) is greater than a maximum blood pressure of a patient in which the catheter assembly 10 is placed. The patient's blood pressure thus keeps the valve 16 closed, preventing blood from leaking through the valve 16. However, when a blood collection device, such as a syringe, is connected to the catheter hub 12, suction applied with the blood collection device is sufficient to overcome the second cracking pressure to enable blood collection through the catheter hub 12. In certain embodiments, the second cracking pressure may be in the range from about 400 mmH2O to about 600 mmH2O.

The valve 16 of the present catheter assembly 10 advantageously provides low hydrodynamic resistance (and a low cracking pressure) in the proximal-to-distal flow direction FP→D (FIG. 5A). This advantageous property results from at least one of several structural characteristics of the valve 16. For example, as discussed above, the diaphragm 64 of the valve 16 includes a conical shape with the apex 106 of the diaphragm 64 lying in a plane 108 distal of a plane 110 defined by the base 112 of the diaphragm 64 (FIG. 5A). With reference to FIG. 5, the diaphragm 64 also has a very slight thickness T. In certain embodiments, a thickness T of the diaphragm 64 may be in the range from about 0.2 mm to about 1 mm. Further, with reference to FIG. 2, the slits 78 that define the boundaries of the flaps 80 each have a length L that is large relative to the diameter D of the diaphragm 64. This property results in a large surface area for each flap 80 relative to the diameter D of the diaphragm 64. In certain embodiments, a length of each slit 78 may be in the range from about 1 mm to about 14 mm, and a diameter of the diaphragm 64 may be in the range from about 2 mm to about 15 mm.

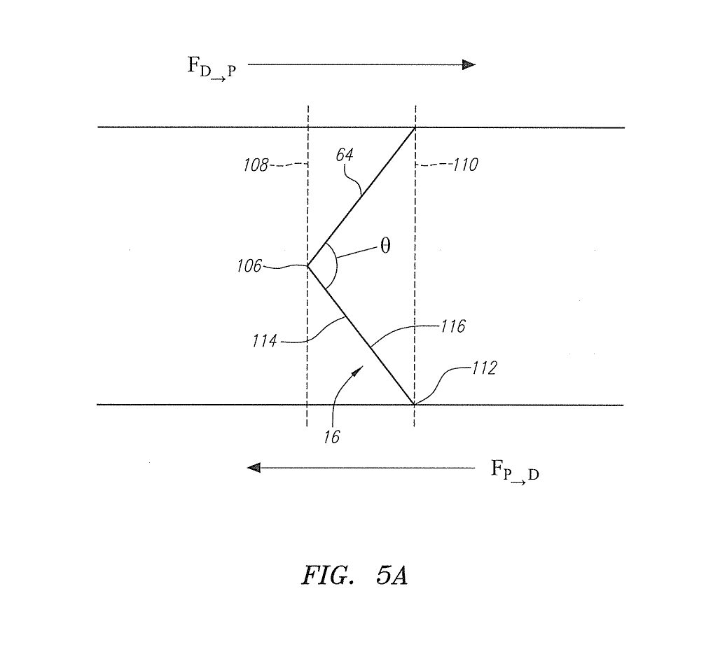

FIG. 6 illustrates another embodiment of the present valved catheter assemblies. The catheter assembly 120 of FIG. 6 has many structural and functional similarities to the valved catheter assembly 10 of FIGS. 1-5. Accordingly, the following discussion will focus on the aspects of the catheter assembly 120 of FIG. 6 that differ from the embodiment of FIGS. 1-5.

The assembly 120 of FIG. 6 includes an abutment member 122 interposed between the proximal catheter hub element 28 and the valve 124. In the illustrated embodiment, the abutment member 122 is shaped as an annular disk having a central opening 126. The abutment member 122 bears against an annular portion of the proximal surface 128 of the valve diaphragm 130. The abutment member 122 restricts the deflection of the diaphragm 130 in the proximal direction, such as during aspiration through the catheter assembly 120. More particularly, the abutment member 122 resists or prevents flexure of the annular portion of the diaphragm 130 that directly contacts the abutment member 122. Thus, the abutment member 122 reduces the effective diameter of the diaphragm 130 for aspiration to the diameter of the central opening 126 in the abutment member 122. Since the cracking pressure of the valve 124 is inversely related to the diameter of the diaphragm 130 (i.e. the smaller the diameter the greater the cracking pressure), the abutment member 122 increases the cracking pressure of the valve 124 for aspiration. The abutment member 122 does not, however, significantly affect the cracking pressure of the valve 124 for infusion, because the abutment member 122 does not restrict the deflection of the diaphragm 130 in the distal direction, as occurs during infusion through the catheter assembly 120. The abutment member 122 may, however, slightly increase the cracking pressure of the valve 124 for infusion by reducing the exposed area of the proximal surface 128 of the diaphragm 130, but this effect is likely to be small.

The abutment member 122 may be constructed of a material that is substantially rigid. A rigid abutment member 122 would provide little if any yield in response to the diaphragm 130 bearing against the abutment member 122 during aspiration, thereby increasing the cracking pressure of the valve 124 for aspiration. Alternatively, the abutment member 122 may be constructed of a material that has at least some flexibility or resilience. Such an abutment member 122 would provide some yield in response to the diaphragm 130 bearing against the abutment member 122 during aspiration, thereby increasing the cracking pressure of the valve 124 for aspiration to a lesser extent than a rigid abutment member 122. The flexibility or resilience of the abutment member 122 could thus be tailored to provide a desired aspiration cracking pressure for the valve 124. Other techniques for tailoring the aspiration cracking pressure for the valve 124 include varying the dimensions of the abutment member 122, such as increasing or decreasing the diameter of the central opening 126 in the abutment member 122.

With continued reference to FIG. 6, the illustrated valve 124 further includes an annular lip 132 circumscribing its distal surface 134. The annular lip 132 bears against the shoulder 68 of the distal catheter hub element 30, thereby creating a gap 136 between the shoulder 68 and the diaphragm 130. The gap 136 provides space into which the diaphragm 130 may deflect during infusion, which enhances the advantageous differential cracking pressures for the valve 124, i.e. the aspiration cracking pressure is significantly higher than the infusion cracking pressure.

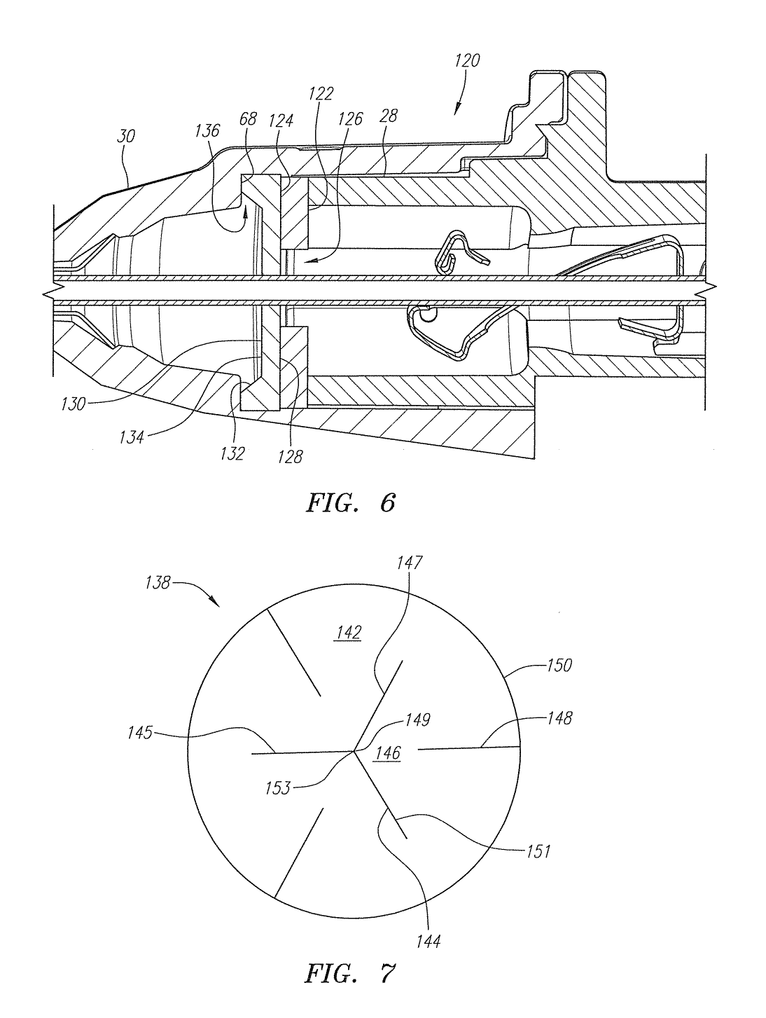

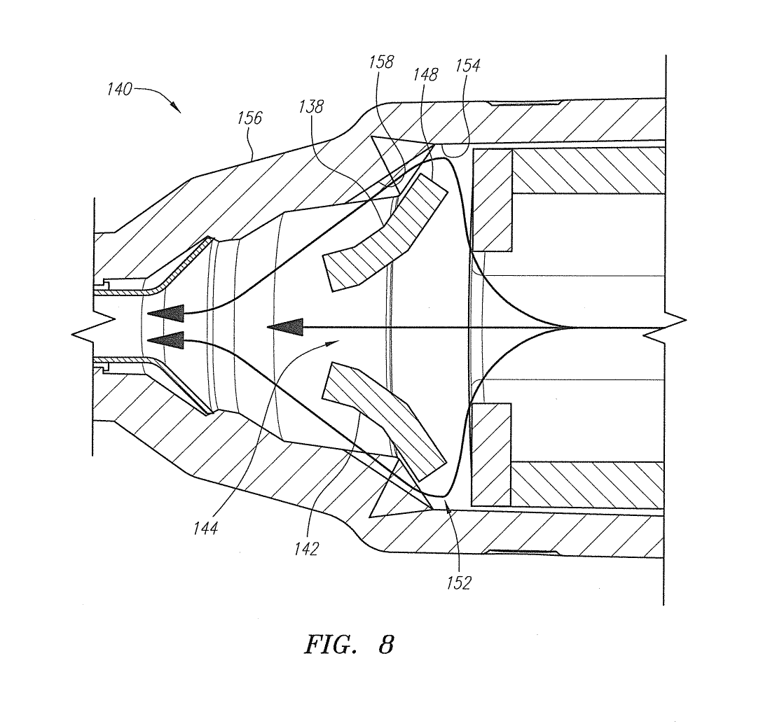

FIG. 7 illustrates another embodiment of a valve 138 configured for use with the present valved catheter assemblies. FIG. 8 illustrates another embodiment of the present valved catheter assemblies, including the valve 138 of FIG. 7 and illustrating the configuration of the assembly 140 during an infusion procedure. While the valve 138 shown in FIG. 7 is described below with reference to the catheter assembly 140 of FIG. 8, the valve 138 shown in FIG. 7 is configured for use in any of the valved catheter assemblies described herein, including the embodiments of FIGS. 1-6. Further, the valves described above, including the valves 16, 124 shown in FIGS. 1-6, are configured for use in any of the valved catheter assemblies described herein, including the embodiment of FIG. 8.

With reference to FIG. 7, the valve 138 includes a diaphragm 142 having a first plurality of slits, or central slits 144, that define edges of flaps 146. In the valve 138 of FIG. 7, the central slits 144 do not all intersect at the same point. Rather, first and second ones 145, 147 of the central slits 144 intersect one another at or near a center 149 of the diaphragm 142. A third one 151 of the central slits 144 intersects the first central slit 145 at a point 153 spaced from the center 149 of the diaphragm 142. The first and third central slits 145, 151 do not intersect one another. Locations, lengths, and whether the slits intersect can be selected or adjusted to change the minimum cracking and closing pressure of the valve.

The valve 138 further includes a second plurality of slits, or peripheral slits 148. Each of the peripheral slits 148 extends from an outer edge 150 of the valve diaphragm 142 inward toward a center of the diaphragm 142. However, each of the peripheral slits 148 stops short of the center of the diaphragm 142. Further, the second plurality of slits includes three peripheral slits 148, and each peripheral slit 148 is spaced approximately 120° from the other peripheral slits 148. Further, each peripheral slit 148 is offset by approximately 60° with respect to the central slits 144. In other words, each peripheral slit 148 bisects an angle formed by adjacent ones of the central slits 144.

With reference to FIG. 8, the peripheral slits 148 provide additional flow paths 152 for fluid infused through the catheter assembly 140. When the valve 138 reaches its cracking pressure during infusion, the infused fluid flows through both the central slits 144 and through the peripheral slits 148, as shown in FIG. 8. As also shown in FIG. 8, the inner surface 154 of the distal catheter hub element 156 includes a chamfer 158 located just distal of the valve 138. The chamfer 158 provides space into which the deflected diaphragm 142 moves during infusion, and enhances the flushability of the valve 138 to reduce blood stagnation, as described further below.

The present valved catheter assemblies advantageously reduce or eliminate dead spaces, because no pusher member is used to open the diaphragm. In catheter assemblies that use a pusher member to open the diaphragm, liquid tends to collect and stagnate in the small spaces between the pusher member and the inner wall of the catheter hub. In the case of infused medication, stagnant liquid in dead spaces can result in less than a desired quantity of medication being delivered to the patient. The present valved catheter assemblies reduce or eliminate the prevalence of this disadvantageous scenario by reducing or eliminating dead spaces.

Further, the valve 138 illustrated in FIG. 7 advantageously enables fluid flow through portions of the diaphragm 142 spaced radially away from the center of the diaphragm 142. As shown in FIG. 8, this peripheral flow flushes fluid, such as blood, from the area around the perimeter of the valve 138, thereby further reducing dead space within the catheter assembly 140. The chamfer 158 enhances fluid flow at the periphery of the valve 138 to enhance flushing and rinsing of blood from the peripheral areas around the valve 138 during infusion, which helps to avoid blood stagnation.

The various components of the present catheter assemblies preferably comprise various medical grade materials, such as polymers and/or metals. For example, and without limitation, certain components, such as the catheter, the catheter hub, the needle hub, and/or the abutment member may comprise polymers, such as nylon, polyethylene, polypropylene, polyurethane, ethylene-vinyl acetate (EVA), polyether block amide (PEBAX), acrylonitrile butadiene styrene (ABS), polyether ether ketone (PEEK), polytetrafluoroethylene (PTFE), thermoplastic polyetherimide (ULTEM), etc. For example, and without limitation, certain components, such as the needle, and/or the needle tip protector, may comprise metals, such as stainless steel, titanium, cobalt-chromium, etc.

As described above, the various embodiments of the present catheter assemblies provide several advantages. For example, the catheter assemblies provide differential cracking pressures based on the direction of flow through the catheter assemblies, with a lesser cracking pressure in the inflow direction and a greater cracking pressure in the outflow direction. The inflow cracking pressure is also very low, including close to zero in some embodiments, which facilitates easy infusion of fluids to the patient. The outflow cracking pressure is great enough to prevent leakage of blood through the catheter assemblies, but low enough to enable easy blood draw. When the needle is disposed within the catheter assemblies, such as during a venipuncture procedure, very small gaps between adjacent flaps in the valve enable outflow (venting) of air while blocking outflow of blood. The catheter assemblies thus facilitate reliable catheter placement by allowing the operator to verify vein access through visible flashback.

The above description presents various embodiments of the present invention, and the manner and process of making and using them, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention.

What is claimed is:

1. A catheter assembly, comprising:
a catheter hub defining an interior cavity;
a catheter extending distally from the catheter hub, the catheter including a catheter lumen;
a valve disposed within the interior cavity of the catheter hub, the valve including a conically shaped diaphragm with at least one slit defining at least two flaps;
a needle hub engaging a proximal end of the catheter hub; and
a needle extending distally from the needle hub, through the catheter hub, through the valve, and through the catheter lumen, the needle having a sharp distal tip.

2. The catheter assembly of claim 1, wherein an apex of the diaphragm lies in a plane distal of a plane defined by a base of the diaphragm.

3. The catheter assembly of claim 1, wherein when the needle extends through the valve a gap between the valve flaps has sufficient width to allow passage of gaseous particles but has insufficient width to allow passage of liquid particles.

4. The catheter assembly of claim 1, wherein after the needle is withdrawn from the valve, the valve has a first cracking pressure in the proximal-to-distal flow direction and a second cracking pressure in the distal-to-proximal flow direction, and the first and second cracking pressures are different.

5. The catheter assembly of claim 4, wherein the second cracking pressure is greater than the first cracking pressure.

6. The catheter assembly of claim 4, wherein the second cracking pressure is greater than a maximum blood pressure of a patient in which the catheter assembly is placed.

7. The catheter assembly of claim 1, wherein the valve includes an annular flange at a periphery of the valve.

8. The catheter assembly of claim 7, wherein the flange extends proximally of the diaphragm.

9. The catheter assembly of claim 7, wherein a proximal face of the valve includes a circular lip positioned radially inward of the flange.

10. The catheter assembly of claim 1, wherein the catheter hub includes a proximal hub element and a distal hub element formed as discrete components and fitted together.

11. The catheter assembly of claim 10, wherein a periphery of the valve is interposed between the proximal hub element and the distal hub element of the catheter hub.

12. The catheter assembly of claim 11, wherein a proximal face of the valve includes a circular lip that bears against an annular distal face of the proximal hub element of the catheter hub.

13. The catheter assembly of claim 10, wherein the valve includes an annular flange at a periphery of the valve, and the annular flange overlaps and surrounds a distal portion of the proximal hub element of the catheter hub.

14. The catheter assembly of claim 13, wherein the annular flange is interposed between the proximal hub element and the distal hub element of the catheter hub as measured along the direction perpendicular to a longitudinal axis of the catheter.

15. The catheter assembly of claim 1, wherein the diaphragm includes three slits extending radially outward from a center of the diaphragm and defining three flaps.

16. The catheter assembly of claim 1, wherein the diaphragm further comprises a plurality of reinforcing ribs located, at least in part, on the flaps.

17. The catheter assembly of claim 1, further comprising a needle tip protector disposed within the interior cavity of the catheter hub.

18. A catheter assembly, comprising:
a catheter hub defining an interior cavity;
a catheter extending distally from the catheter hub, the catheter including a catheter lumen; and
a valve disposed within the interior cavity of the catheter hub, the valve including a diaphragm with at least one slit defining at least two flaps;
wherein the valve has a first cracking pressure in the proximal-to-distal flow direction and a second cracking pressure in the distal-to-proximal flow direction, and the first and second cracking pressures are different.

19. The catheter assembly of claim 18, wherein the second cracking pressure is greater than the first cracking pressure.

20. The catheter assembly of claim 18, wherein the second cracking pressure is greater than a maximum blood pressure of a patient in which the catheter assembly is placed.

21. The catheter assembly of claim 18, wherein a central portion of the diaphragm extends distally with respect to a periphery of the diaphragm.

22. The catheter assembly of claim 18, wherein the valve includes an annular flange at a periphery of the valve.

23. The catheter assembly of claim 22, wherein the flange extends proximally of the diaphragm.

24. The catheter assembly of claim 22, wherein a proximal face of the valve includes a circular lip positioned radially inward of the flange.

25. The catheter assembly of claim 18, wherein the catheter hub includes a proximal hub element and a distal hub element formed as discrete components and fitted together.

26. The catheter assembly of claim 25, wherein a periphery of the valve is interposed between the proximal hub element and the distal hub element of the catheter hub.

27. The catheter assembly of claim 26, wherein a proximal face of the valve includes a circular lip that bears against an annular distal face of the proximal hub element of the catheter hub.

28. The catheter assembly of claim 25, wherein the valve includes an annular flange at a periphery of the valve, and the annular flange overlaps and surrounds a distal portion of the proximal hub element of the catheter hub.

29. The catheter assembly of claim 28, wherein the annular flange is interposed between the proximal hub element and the distal hub element of the catheter hub as measured along the direction perpendicular to a longitudinal axis of the catheter.

30. The catheter assembly of claim 18, wherein the diaphragm includes three slits extending radially outward from a center of the diaphragm and defining three flaps.

31. The catheter assembly of claim 18, wherein the diaphragm further comprises a plurality of reinforcing ribs located, at least in part, on the flaps.
